(19) United States
(12) Patent Application Publication
(10) Pub. No.: US 2023/0293912 A1
(43) Pub. Date: Sep. 21, 2023

(54) HARMONIC EXCITATION TISSUE THERAPY

(71) Applicant: Open Water Internet Inc., San Francisco, CA (US)

(72) Inventors: Achal Singh Achrol, Highland, CA (US); Soren Konecky, Alameda, CA (US); Christopher R. Bawiec, San Francisco, CA (US); Brad A. Hartl, San Mateo, CA (US); Ulas Taskin, Delft (NL); Sarah Barreto Ornellas, San Francisco, CA (US)

(21) Appl. No.: 18/122,124

(22) Filed: Mar. 16, 2023

Related U.S. Application Data
(60) Provisional application No. 63/321,056, filed on Mar. 17, 2022.

(51) Int. Cl. A61N 7/00

(52) U.S. Cl. CPC A61N 7/00; A61N 2007/0078; A61N 2007/0052; A61N 2007/0056; A61N 2007/0086

(57) ABSTRACT

One or more ultrasonic transducers are driven to direct a therapeutic ultrasound signal into tissue. The therapeutic ultrasound signal has a resonant ultrasound frequency to harmonically excite and damage abnormal tissue but not harmonically excite healthy tissue.

### CROSS-REFERENCE TO RELATED APPLICATIONS

This application claims priority to U.S. provisional Application No. 63/321,056 filed Mar. 17, 2022, which is hereby incorporated by reference.

### TECHNICAL FIELD

This disclosure relates generally to medical devices and treatments, and in particular to ultrasound.

### BACKGROUND INFORMATION

Conventional cancer treatments include surgical removal, chemotherapy, and radiation. Chemotherapy includes delivering a drug intravenously to shrink or kill cancer cells while radiation utilizes high-energy beams to destroy or suppress the cancer cells. More recently, therapies that deliver an electric field that change polarities to the cancer cells have been shown to suppress division and/or growth of cancer cells. Among the drawbacks to the electric field therapy is the need for electrodes to be applied to the skin close to the targeted area. For tumors located in the head, this typically limits compliance to the therapy since the therapy frequency is typically daily with hours of duration and the skin site for the electrodes may need to be frequently prepared (e.g. shaved).

Cancer stem cells are the most armored cancer cells and don't divide as quickly as other cancer cells. Since the stem cells don't divide very quickly, they don't expose themselves to be affected by electrical fields and radiation. Hence, the electrical field therapy and radiation therapy may suppress growth in cancer cells, but fail to destroy the root cancer stem cells.

### BRIEF DESCRIPTION OF THE DRAWINGS

Nonlimiting and non-exhaustive embodiments of the invention are described with reference to the following figures, wherein like reference numerals refer to like parts throughout the various views unless otherwise specified.

FIG. 1A illustrates a therapy system including processing logic and one or more ultrasound transducers, in accordance with aspects of the disclosure.

FIG. 1B illustrates a user wearing an example head mountable wearable that may include all or a portion of the system of FIG. 1A, in accordance with aspects of the disclosure.

FIG. 1C illustrates a head mountable wearable that includes ultrasonic transducers, in accordance with aspects of the disclosure.

FIG. 2 illustrates an example flow chart illustrating an example oncolysis process, in accordance with aspects of the disclosure.

FIG. 3 illustrates viability results for different ultrasound profiles, in accordance with aspects of the disclosure.

FIG. 4A illustrates cancer cell-laden hydrogel, in accordance with aspects of the disclosure.

FIG. 4B illustrates spheroid-laden hydrogel with cancer cells encapsulated within the spheroids, in accordance with aspects of the disclosure.

FIGS. 5-6 illustrate results of a plurality of brain cancer-derived cell cultures grown as 3D cultures in hydrogel as brain cancer organoids that were screened against ultrasound signals having different ultrasound profiles and control conditions, in accordance with aspects of the disclosure.

FIGS. 7-8 illustrate testing results by Applicant of cell viability in spheriod-laden hydrogel with respect to the mechanical index (MI) of the resonant ultrasound frequency emitted by ultrasound transducers, in accordance with aspects of the disclosure.

FIGS. 9-10 illustrate testing results by Applicant of cell viability in cell-laden hydrogel with respect to the mechanical index (MI) of the resonant ultrasound frequency emitted by ultrasound transducers, in accordance with aspects of the disclosure.

FIG. 11 illustrates healthy cell viability after receiving the resonant ultrasound frequencies provided in FIGS. 9 and 10, in accordance with aspects of the disclosure.

FIG. 12 illustrates an example flow chart illustrating an example process for patient-specific harmonic excitation therapy, in accordance with aspects of the disclosure.

FIG. 13 illustrates an example therapy system that includes a wearable, a device, and an example anti-tumor agent delivery device, in accordance with aspects of the disclosure.

FIG. 14 illustrates an example flow chart illustrating an example tumor treatment process, in accordance with aspects of the disclosure.

FIG. 15 illustrates an example flow chart illustrating an example tumor treatment process, in accordance with aspects of the disclosure.

### DETAILED DESCRIPTION

Embodiments of systems, devices, and methods of delivering harmonic excitation tissue therapy are described herein. In the following description, numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Throughout this specification, several terms of art are used. These terms are to take on their ordinary meaning in the art from which they come, unless specifically defined herein or the context of their use would clearly suggest otherwise.

Systems, devices, and methods of this disclosure include ultrasound transducers directing therapeutic ultrasound signals to abnormal tissue (e.g. cancer cells such as glioblastoma) at a resonant ultrasound frequency to harmonically excite and consequently damage/destroy the abnormal tissue. Harmonic excitation techniques take advantage of the fragility of the abnormal tissue due to the stiffer properties of the abnormal tissue when compared to healthy tissue. The stiffer properties of the abnormal cells may be due to the cytoskeletal weakness of rapidly dividing cells. The abnormal cells may differ relative to surrounding healthy tissue in aspects such as (1) overall cell size; (2) presence of nuclear atypia; (3) different nuclear-to-cytoplasm ratios; (4) fragility of cytoskeleton networks; and (5) cell membrane characteristics. The disclosed harmonic excitation techniques take advantage of these differences to allow a therapeutic ultrasound signals at the resonant ultrasound frequency (tuned to destroy the abnormal tissue) to be an effective non-invasive tumor therapy that causes cell lysis and apoptosis in abnormal cells while healthy cells are not negatively affected.

Yet another potential benefit of the disclosed harmonic excitation techniques is the disintegration of the abnormal tissue releases the tumor antigens into the body that can lead to an immune activation. Therefore, the harmonic excitation of cancer cells also may function as an in situ vaccination. The in situ vaccination can then be combined with systemic immunotherapies to improve the efficacy with a kindling effect.

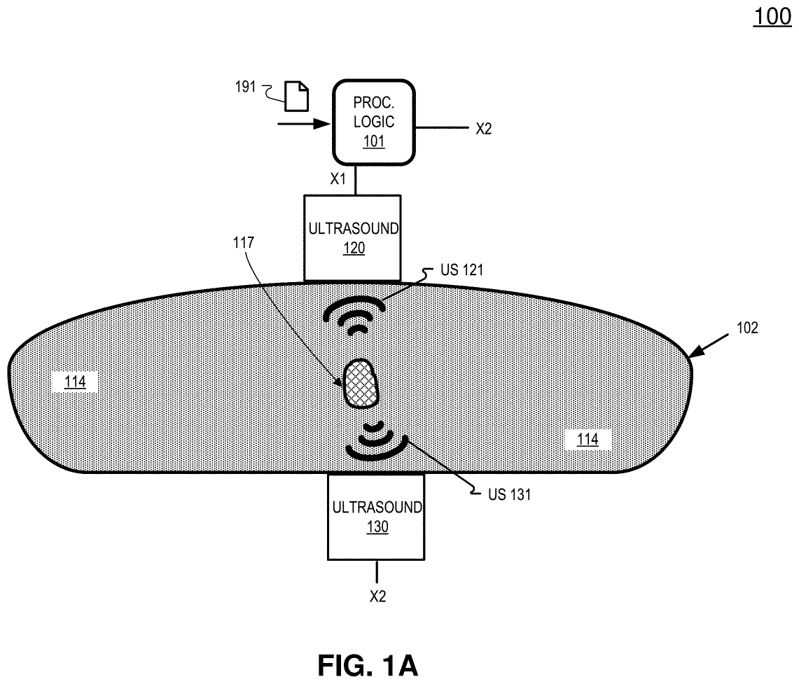

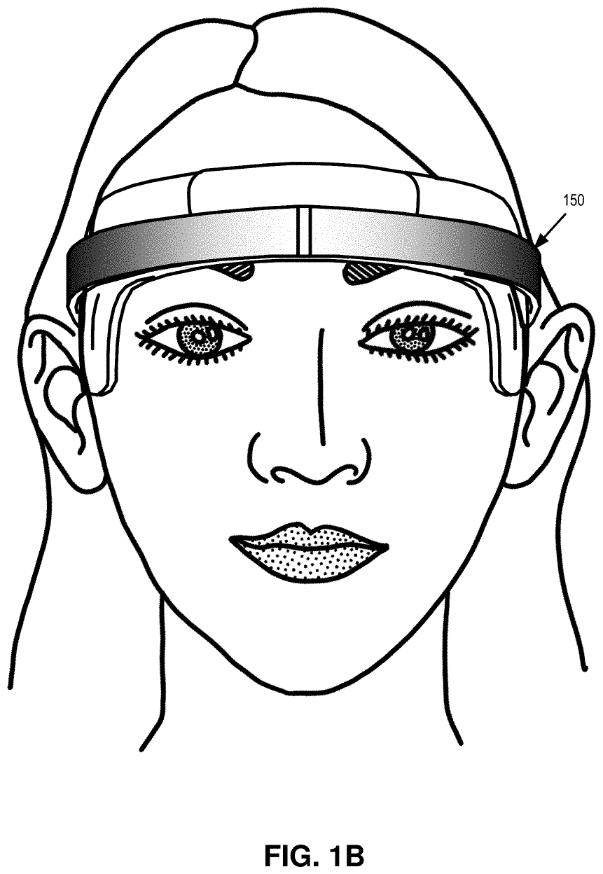

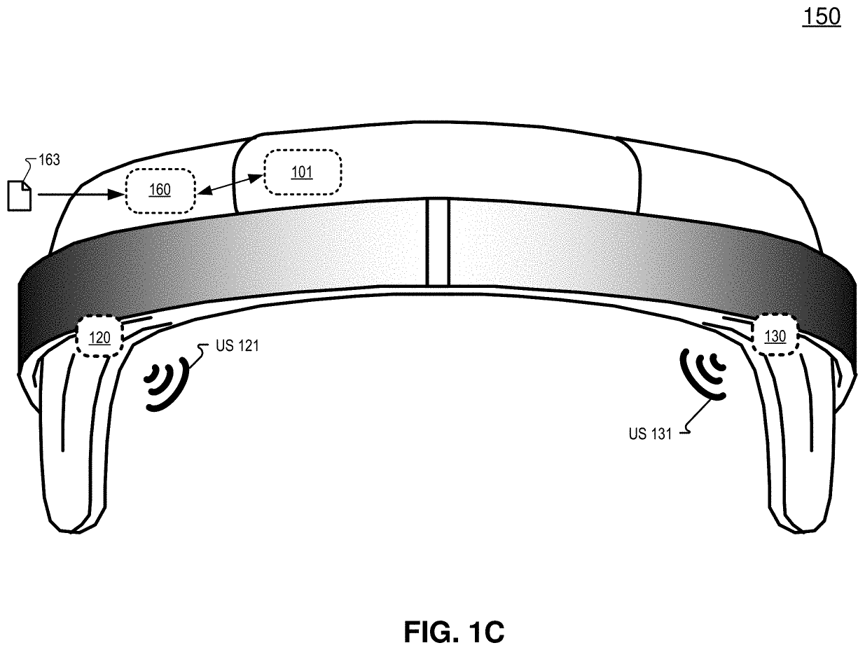

In implementations of the disclosure, the harmonic excitation techniques are paired with an anti-tumor agent therapy such as an immunotherapy or large molecule anti-tumor biologic agent. Example anti-tumor agent therapies may include (1) an antibody-based anti-tumor agent; (2) a monoclonal antibody; (3) bispecific antibody, (4) antibody-drug conjugate; (5) chimeric antigen receptor (CAR); (6) T cell therapy; (7) engineered T cell receptor (TCR) therapy; (8) adoptive T cell therapy; (9) natural killer (NK) cell therapy, or (10) dendritic cell therapy, for example. Pairing the anti-tumor agent with the therapeutic ultrasound signal may increase neoantigen release from the tumor to prime an immune response from the patient. Importantly, synergistically pairing the anti-tumor agent therapy with the harmonic excitation may yield better results than the treatments provide separately. These and other implementations will be described in FIGS. 1A-15.

FIG. 1A illustrates a therapy system 100 including processing logic 101 and one or more ultrasound transducers, in accordance with aspects of the disclosure. In the particular implementation of FIG. 1, system 100 includes ultrasonic transducer 120 configured to emit therapeutic ultrasonic signal 121 and ultrasonic transducer 130 configured to emit therapeutic ultrasonic signal 131. Ultrasonic transducer 120 and ultrasonic transducer 130 may include a plurality of ultrasonic emitters for emitting the therapeutic ultrasonic signals 121/131 and may include one or more ultrasonic receivers to sense an ultrasonic return signal. Ultrasonic transducers 120 and 130 may include an array of steerable ultrasound emitters that can be driven to provide beam-forming capability of the therapeutic ultrasound signal(s), in some implementations. System 100 may include any number of ultrasonic transducers to deliver therapeutic ultrasound signals to tissue.

Some or all of the components of system 100 may be included in a wearable device or article that is configured to have the ultrasonic emitters contact the skin of a patient so that the therapeutic ultrasonic signal 121 may propagate into the tissue 102. The wearable device may be configured to be secured to a head, arm, or other body part. Tissue 102 includes healthy tissue 114 and abnormal tissue 117. Abnormal tissue 117 may be a tumor or cancer. Processing logic 101 is configured to drive ultrasound transducer 120 via communication channel X1 and configured to drive ultrasound transducer 130 via communication channel X2. In an implementation, processing logic 101 is configured to drive the one or more ultrasonic transducers to direct a therapeutic ultrasound signal (e.g. signal 121 and/or 131) into tissue 102 brought into contact with the ultrasonic transducers. The therapeutic ultrasound signal(s) have a resonant ultrasound frequency to harmonically excite and damage abnormal tissue 117 but not harmonically excite healthy tissue 114 even though the therapeutic ultrasound signal(s) may propagate through both healthy tissue 114 and abnormal tissue 117.

Processing logic 101 may be configured to drive the ultrasonic transducers 120/130 to scan the therapeutic ultrasound signal(s) through a brain to provide the therapeutic ultrasound signal(s) to substantially all of the tissue in the brain. Driving the ultrasonic transducers may include driving the ultrasonic emitters of the ultrasonic transducers with beam forming pattern signals to achieve the scan. In some implementations, the ultrasonic transducers may be anatomically co-registered with a head of a user/wearer so that ultrasonic emitters can be driven the provide the therapeutic ultrasound signal(s) to substantially all of the tissue in the brain.

Processing logic 101 may drive one or more ultrasonic emitters to emit a therapeutic ultrasonic signal at a resonant ultrasound frequency between 50 kHz and 1 MHz to harmonically excite abnormal tissue 117 and therefore cause lysis of abnormal tissue 117. Harmonic excitation of abnormal tissue 117 to cause lysis is analogous to the ability of an opera singer to break a wine glass with sound waves. In an implementation, the resonant ultrasound frequency selected is between 50 kHz and 700 kHz. In an implementation, the resonant ultrasound frequency selected is between 120 kHz and 230 kHz. In an implementation, the resonant ultrasound frequency selected is between 500 kHz and 670 kHz. In an implementation, the resonant ultrasound frequency selected is between 70 kHz and 90 kHz. In an implementation, the resonant ultrasound frequency selected is between 75 kHz and 85 kHz. In an implementation, the resonant ultrasound frequency selected is approximately 80 kHz. In an implementation, the resonant ultrasound frequency selected is between 95 kHz and 105 kHz. In an implementation, the resonant ultrasound frequency selected is approximately 100 kHz. In an implementation, the resonant ultrasound frequency selected is between 145 kHz and 155 kHz. In an implementation, the resonant ultrasound frequency selected is approximately 150 kHz. The resonant ultrasound frequency may be selected based on the type of cancer being targeted (e.g. ovarian cancer of brain cancer).

The pulse duration of the emitted therapeutic ultrasound signal may be between 1 ms and 500 ms or even 1 us and 1 second. In an implementation, the therapeutic ultrasound signal(s) are directed into the tissue in bursts that are 30 ms or longer. In an implementation, the therapeutic ultrasound signal(s) are directed into the tissue in bursts that are 40 ms or longer. In an implementation, the therapeutic ultrasound signal(s) are directed into the tissue in bursts that are approximately 40 ms. The duty cycle of the emitted therapeutic ultrasound signal may be between 1% and 50% or even 0.1% to 100% and the pulse repetition frequency may between 0.1 Hz to 10 kHz. The beam width of the emitted therapeutic ultrasound signal at abnormal tissue 117 is dependent on the resonant ultrasound frequency and may be between 1 mm and 15 mm. In some implementations, the beam width at abnormal tissue 117 is between 8 mm and 10 mm. In an implementation, a max peak rarefactional pressure of the emitted therapeutic ultrasound signal is 2 MPa.

In an implementation, processing logic 101 may receive a medical image 191 and determine a location of abnormal tissue 117 and a resonant ultrasound frequency of the abnormal tissue 117 based on the medical image 191. The medical image 191 may be an MRI, a CT scan, or otherwise. In other implementations, the resonant ultrasound frequency to be used for the ultrasonic emitters to direct to the abnormal tissue 117 is transmitted (wired or wirelessly) to processing logic 101.

In an implementation, processing logic 101 drives one or more ultrasound transducers (e.g. 120 and/or 130) to direct the therapeutic ultrasound signal (e.g. 121 and/or 131) to a location of abnormal tissue 117. Processing logic 101 may drive an array of ultrasound emitters to achieve beam forming to direct the therapeutic ultrasound signal to the location of abnormal tissue 117. Processing logic 101 may selectively drive some ultrasonic emitters to direct the therapeutic ultrasound signal(s) to the abnormal tissue 117 based on a proximity of one or more of the ultrasound emitters to the abnormal tissue 117. In some implementations, processing logic 101 selectively drives ultrasonic emitters to direct the therapeutic ultrasound signal(s) to the abnormal tissue 117 based on medical image 191 where medical image 191 includes the location of the abnormal tissue 117.

In implementations, processing logic 101 drives one or more ultrasound emitters (e.g. 120 and/or 130) to emit a chirp therapeutic ultrasound signal where the frequency increases (up-chirp) or decreases (down-chirp) with time. In implementations, processing logic 101 drives one or more ultrasound emitters (e.g. 120 and/or 130) to emit a frequency swept therapeutic ultrasound signal. Changing the frequency of the therapeutic ultrasound signal in these ways may increase the efficacy of the treatment by increasing the probability that the therapeutic ultrasound signal is tuned to the frequency that harmonically excites the abnormal tissue 117.

FIG. 1B illustrates a user wearing an example head mountable wearable 150 that may include all or a portion of the system 100 of FIG. 1A, in accordance with implementations of the disclosure. FIG. 1C illustrates head mountable wearable 150 that includes ultrasonic transducers 120 and 130. Head mountable wearable 150 may include an input interface 160 configured to receive an anti-tumor agent alert 163. Input interface 160 may include a wireless radio configured to transmit and/or receive wireless data, for example. Anti-tumor agent alert 163 may be a wireless message, in some implementations. Processing logic is communicatively coupled (wired or wirelessly) to input interface 160.

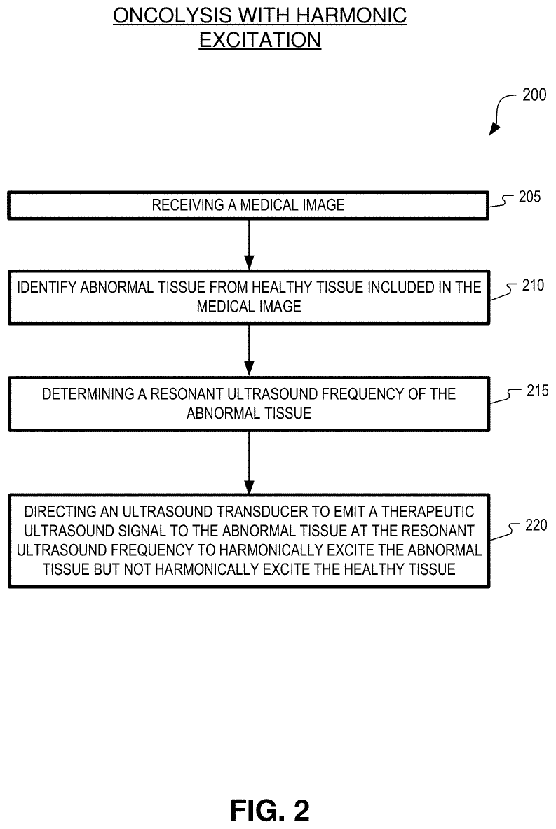

FIG. 2 illustrates an example flow chart illustrating an example oncolysis process 200, in accordance with implementations of the disclosure. The order in which some or all of the process blocks appear in process 200 should not be deemed limiting. Rather, one of ordinary skill in the art having the benefit of the present disclosure will understand that some of the process blocks may be executed in a variety of orders not illustrated, or even in parallel. All or a portion of process 200 may be executed by wearable 150, for example.

In process block 205, a medical image (e.g. image 191) is received. The medical image 191 may be an MRI, a CT scan, or otherwise.

In process block 210, abnormal tissue (e.g. tissue 117) is identified from healthy tissue (e.g. tissue 114) included in the medical image.

In process block 215, a resonant ultrasound frequency is determined for the abnormal tissue.

In process block 220, an ultrasound transducer is directed to emit a therapeutic ultrasound signal to the abnormal tissue at the resonant ultrasound frequency to harmonically excite the abnormal tissue (but not harmonically excite the healthy tissue).

In an implementation of process 200, the therapeutic ultrasound signal propagates through the healthy tissue and the abnormal tissue.

In an implementation of process 200, determining the resonant ultrasound frequency of the abnormal tissue includes identifying the abnormal tissue as a particular tumor type from a group of tumor types (e.g. ovarian cancer or brain cancer) and assigning a particular ultrasound frequency as the resonant ultrasound frequency where the particular ultrasound frequency is associated with harmonic excitation of the particular tumor type. For example, if ovarian cancer has a particular resonant ultrasound frequency, the resonant ultrasound frequency associated with ovarian cancer cells will be selected from a group of resonant ultrasound frequencies when ovarian cancer is identified as the abnormal tissue. And if brain cancer is identified as the abnormal tissue, the resonant ultrasound frequency associated with brain cancer cells will be selected from a group of resonant ultrasound frequencies. The categories of cancer cells and their associated resonant ultrasound frequencies may be stored in a look up table in memory accessible by (or included in) processing logic 101, for example. Experiments by Applicant indicated that approximately 100 kHz is the most effective resonant ultrasound frequency for 1 glioblastoma cancers and approximately 150 kHz is the most effective resonant ultrasound frequency for 2 glioblastoma cancers. Hence, the resonant ultrasound frequency may be adjusted based on identification of the abnormal tissue.

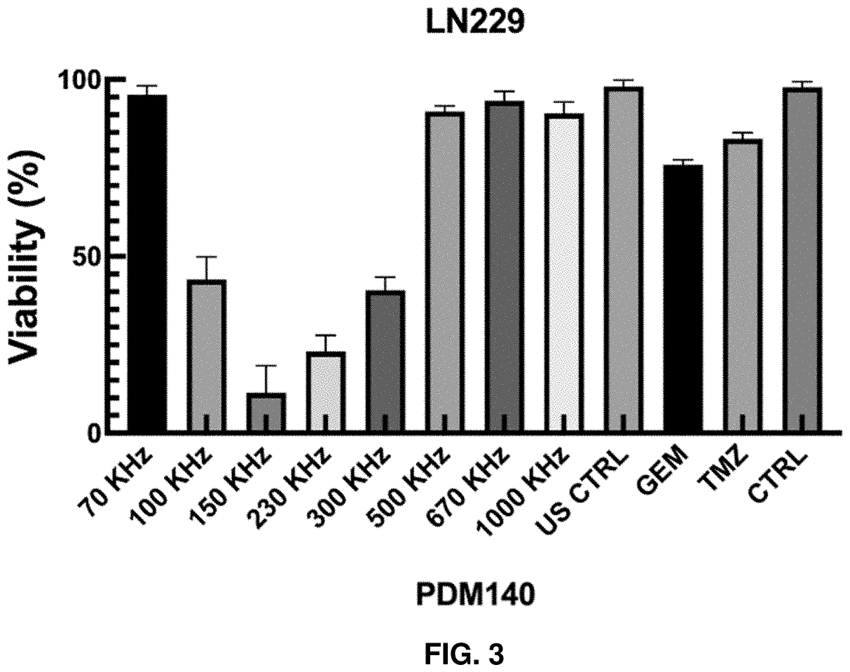

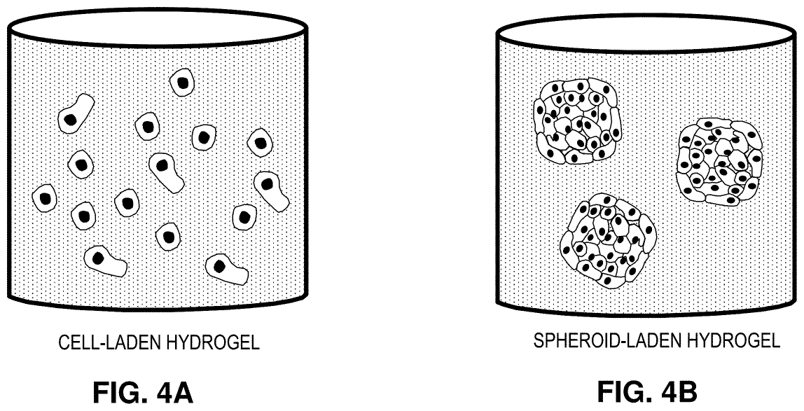

FIG. 3 illustrates viability results for different ultrasound profiles, in accordance with implementations of the disclosure. Brain cancer-derived cell cultures were grown as 3D cultures in hydrogel (i.e. “brain cancer organoids”) for in vitro screening. FIG. 4A illustrates cancer cell-laden hydrogel and FIG. 4B illustrates spheroid-laden hydrogel with cancer cells encapsulated within the spheroids. Thus, a tissue sample of abnormal tissue (e.g. a tumor) can be grown into 3D cell cultures with extracellular matrixes for testing and experimentation purposes. FIG. 3 shows results for brain cancer organoids being exposed to different ultrasound beam treatments having different ultrasound profiles consisting of a range of carrier frequencies in order to identify which frequency produced the greatest cell death (i.e. loss of viability) via harmonic excitation of the target cancer cells. These brain cancer organoids were created using glioblastoma cells called LN-229, which can be provided by American Type Culture Collection of the state of Virginia in the United States. This same process can be repeated across other cell cultures as well as with patient-derived cancer cell lines obtained from surgical resection of a tumor to determine the optimal resonant frequency for each cancer in a personalized, patient-specific manner.

In the illustrated test results, the ultrasound beam had sonication parameters that consisted of a mechanical index (MI) of 100% and a treatment regimen consisting of 40 ms bursts at a 10% duty cycle (i.e. 300 bursts) for a total treatment exposure time of 120 seconds. These parameters were held constant while the frequency was changed across each organoid to screen for the optimal resonant frequency for cancer cell lysis (i.e. greatest loss of viability). Additional brain cancer organoids were exposed to control conditions (CTRL in FIG. 3) for comparison, including general control organoids (no treatment), organoids labeled ultrasound control were placed within the same ultrasound set up but received a sham treatment (no ultrasound beam), as well as organoids treated with the chemotherapy drug Gemcitabine (GEM), and organoids treated with the chemotherapy drug Temozolomide (TMZ). Prior to imaging the brain cancer organoids in each condition to measure their response, a cell viability marker may be introduced to assist in imaging the brain cancer organoids. The cell viability markers and their applications are known in the industry. The bar graph of FIG. 3 illustrates the viability of each of the plurality of brain cancer organoids with respect to the ultrasound frequency or control condition that was directed to the brain cancer organoids. Thus, the results of FIG. 3 indicate approximately 150 kHz for a resonant ultrasound frequency may be the most effective frequency for a particular category of tumor.

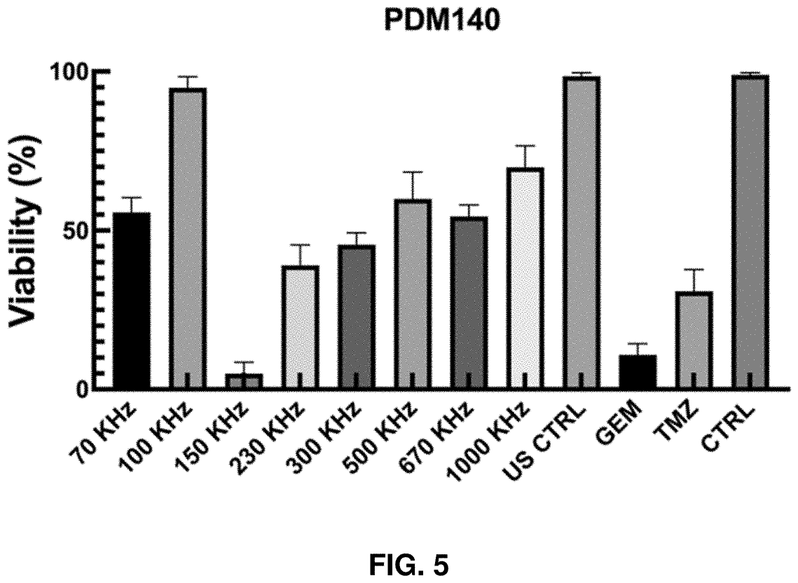

FIG. 5 shows results of a plurality of brain cancer-derived cell cultures grown as 3D cultures in hydrogel as brain cancer organoids that were screened against ultrasound signals having different ultrasound profiles and control conditions, in accordance with aspects of the disclosure. These brain cancer organoids were created using glioblastoma cells called PDM140, which can also be provided by American Type Culture Collection of the state of Virginia in the United States. Other cell cultures may also be used. In the illustrated test results, the ultrasound beam had sonication parameters that consisted of a mechanical index (MI) of 100% and a treatment regimen consisting of 40 ms bursts at a 10% duty cycle (i.e. 300 bursts) for a total treatment exposure time of 120 seconds. These parameters were held constant while the frequency was changed across each organoid to screen for the optimal resonant frequency for cancer cell lysis (i.e. greatest loss of viability). Additional brain cancer organoids were exposed to control conditions for comparison, including general control organoids (no treatment), organoids labeled ultrasound control were placed within the same ultrasound set up but received a sham treatment (no ultrasound beam), as well as organoids treated with the chemotherapy drug Gemcitabine (GEM), and organoids treated with the chemotherapy drug Temozolomide (TMZ). Prior to imaging the brain cancer organoids in each condition to measure their response, a cell viability marker may be introduced to assist in imaging the mature cell clusters. The cell viability markers and their applications are known in the industry. The bar graph of FIG. 5 illustrates the viability of each of the plurality of brain cancer organoids with respect to the ultrasound frequency or control condition that was directed to the brain cancer organoid.

Taking the bar graphs of FIG. 3 and FIG. 5 together, Applicant discovered that an ultrasound frequency of 150 kHz was especially effective at treating the brain cancer organoids under observation. Thus, an ultrasound beam that is optimized to involve an ultrasound frequency of approximately 150 kHz can be used to target certain abnormal tissue. Similarly, based on the result for each particular sample of patient-derived brain cancer organoids, each patient may be prescribed a personalized ultrasound therapy utilizing the most effective ultrasound profile (including a particular resonant frequency) such that the ultrasonic emitters provide a personalized therapy to a patient. In some implementations, the frequency for the ultrasound profile is between 145 kHz and 155 kHz. In some implementations, the frequency for the ultrasound profile is between 140 kHz and 160 kHz. In some implementations, the frequency for the ultrasound profile is between 130 kHz and 170 kHz. In some implementations, the frequency for the ultrasound profile is between 120 kHz and 230 kHz. In some implementations, the frequency for the ultrasound profile is between 90 kHz and 110 kHz. The frequency may change based on the particular tissue received from the patient to personalize the therapy to the patient.

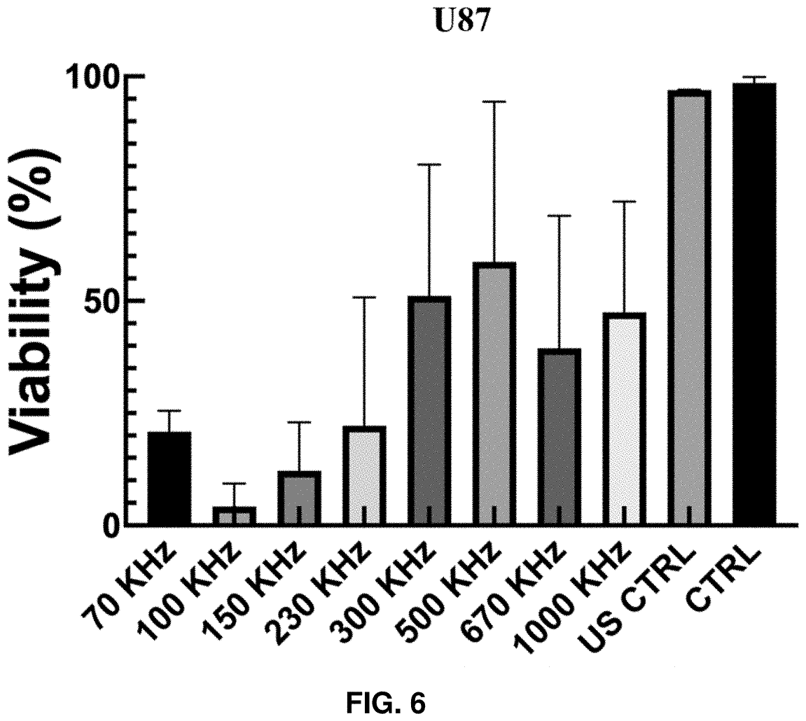

FIG. 6 shows test result of a plurality of brain cancer-derived cell cultures grown as 3D cultures in hydrogel as brain cancer organoids that were screened against ultrasound signals having different ultrasound profiles and control conditions. These brain cancer organoids were created using glioblastoma cells called U87, which can also be provided by American Type Culture Collection of the state of Virginia in the United States. In the illustrated test results, the ultrasound beam had sonication parameters that consisted of a mechanical index (MI) of 100% and a treatment regimen consisting of 40 ms bursts at a 10% duty cycle (i.e. 300 bursts) for a total treatment exposure time of 120 seconds. These parameters were held constant while the frequency was changed across each organoid to screen for the optimal resonant frequency for cancer cell lysis (i.e. greatest loss of viability). Additional brain cancer organoids were exposed to control conditions for comparison, including general control organoids (no treatment), organoids labeled ultrasound control were placed within the same ultrasound set up but received a sham treatment (no ultrasound beam). Prior to imaging the brain cancer organoids in each condition to measure their response, a cell viability marker may be introduced to assist in imaging the mature cell clusters. The bar graph of FIG. 6 illustrates the viability of each of the plurality of brain cancer organoids with respect to the ultrasound frequency or control condition that was directed to the brain cancer organoid. In FIG. 6, 100 kHz was the more effective ultrasound frequency rather than the approximately 150 kHz indicated in FIGS. 3 and 5. Hence, FIG. 6 shows that different kinds of cancer (that have different cell structures) may be susceptible to different ultrasound frequencies since the U87 glioblastoma cells responded differently than PDM140 and LN229 glioblastoma cells.

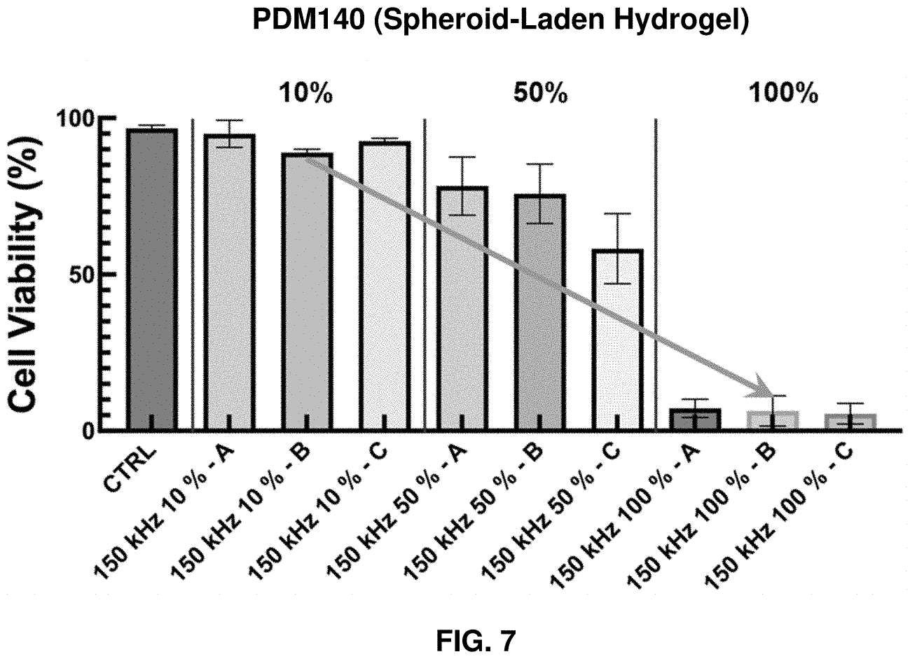

FIG. 7 illustrates testing results by Applicant of cell viability with respect to the mechanical index (MI) of the resonant ultrasound frequency emitted by ultrasound transducers, in accordance with aspects of the disclosure. In particular, FIG. 7 illustrates that a mechanical index of approximately 100% was more effective to reduce cell viability in PDM140 glioblastoma cells than mechanical indexes of 10% or 50%. In some implementations, the mechanical index may be above 95%, but not reach 100%. The cells in the test of FIG. 7 were the spheroid-laden hydrogel cells of FIG. 4B.

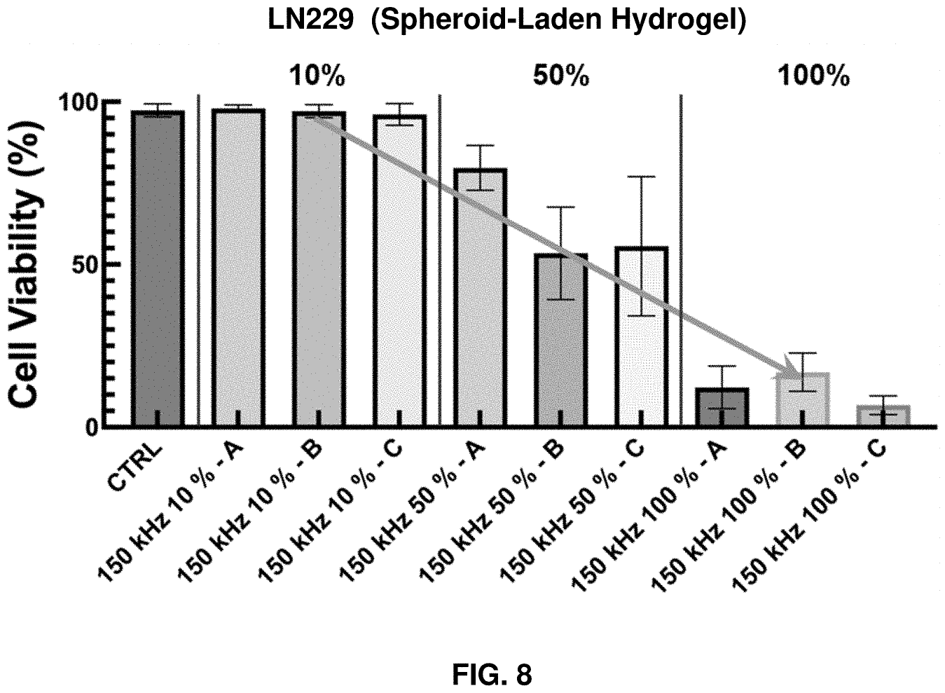

FIG. 8 also illustrates testing results by Applicant of cell viability with respect to the mechanical index (MI) of the resonant ultrasound frequency emitted by ultrasound transducers, in accordance with aspects of the disclosure. In particular, FIG. 8 illustrates that a mechanical index of approximately 100% was more effective to reduce cell viability in LN229 glioblastoma cells than mechanical indexes of 10% or 50%. In some implementations, the mechanical index may be above 95%, but not reach 100%. The cells in the test of FIG. 8 were the spheroid-laden hydrogel cells of FIG. 4B.

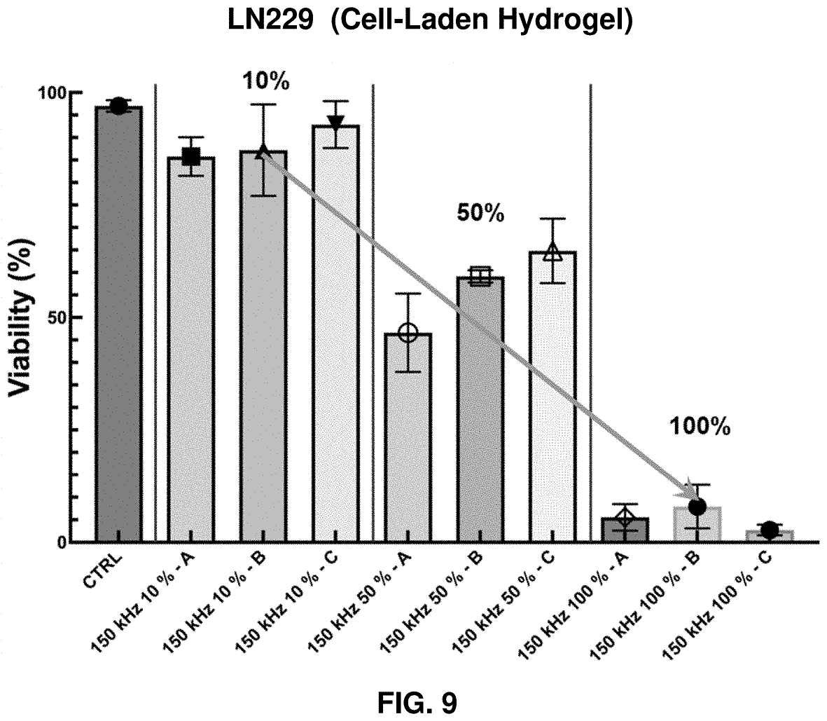

FIG. 9 illustrates testing results by Applicant of cell viability with respect to the mechanical index (MI) of the resonant ultrasound frequency emitted by ultrasound transducers. In particular, FIG. 9 illustrates that a mechanical index of approximately 100% was more effective to reduce cell viability in LN229 glioblastoma cells than mechanical indexes of 10% or 50%. In some implementations, the mechanical index may be above 95%, but not reach 100%. The cells in the test of FIG. 9 were the cell-laden hydrogel cells of FIG. 4A.

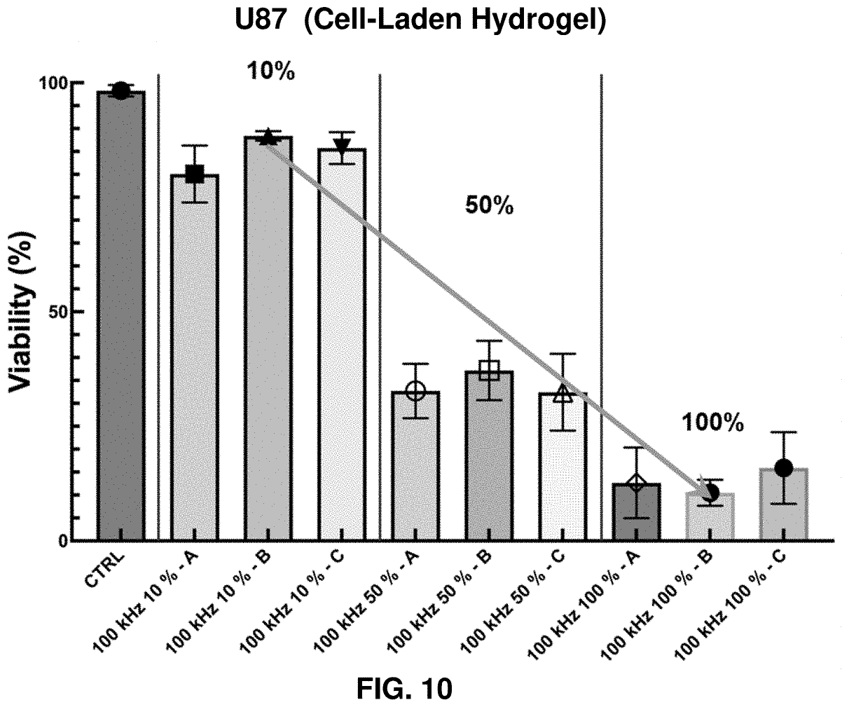

FIG. 10 illustrates testing results by Applicant of cell viability with respect to the mechanical index (MI) of the resonant ultrasound frequency emitted by ultrasound transducers. In particular, FIG. 10 illustrates that a mechanical index of approximately 100% was more effective to reduce cell viability in U87 glioblastoma cells than mechanical indexes of 10% or 50%. In some implementations, the mechanical index may be above 95%, but not reach 100%. The cells in the test of FIG. 10 were the cell-laden hydrogel cells of FIG. 4A.

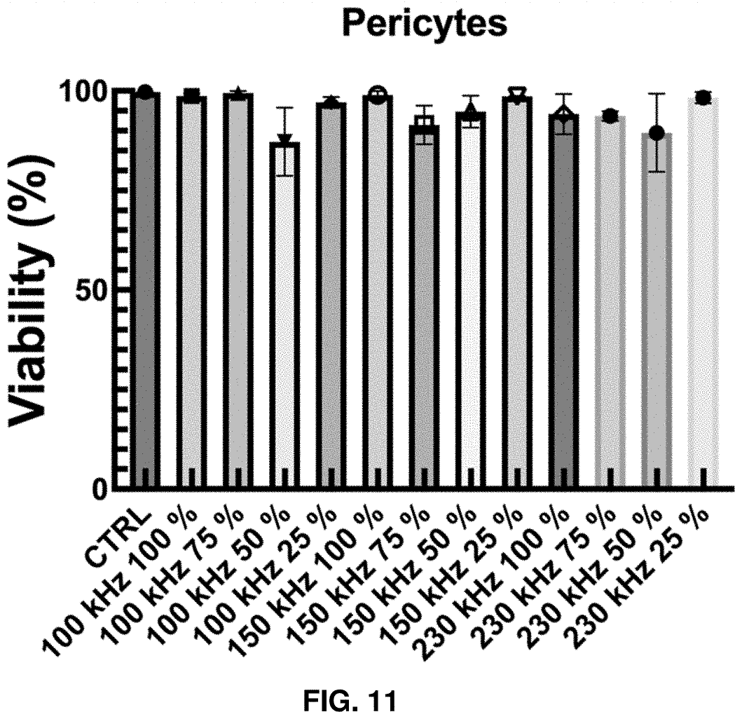

FIG. 11 illustrates healthy cell viability after receiving the resonant ultrasound frequencies provided in FIGS. 9 and 10. When compared with FIGS. 9 and 10, FIG. 11 illustrates selective lysis of cancerous cells of FIGS. 9 and 10 while healthy cells (in this case pericytes) of FIG. 11 are largely spared from lysis (not harmonically excited) and remain viable.

In implementations of the disclosure, a patient-specific therapy is developed to tune the harmonic excitation therapy for a specific person and for the actual abnormal tissue that is the target of the harmonic excitation therapy. As an overview, an abnormal tissue (e.g. tumor) can be collected with a biopsy. Then a variety of ultrasound profiles are directed to portions of the abnormal tissue. The ultrasound profiles that are most effective at killing the tumor cells can then be programmed into a wearable device to deliver therapeutic ultrasound having the ultrasound profile that has been determined to be most effective at killing the actual tumor in question.

This personalized therapy technique may include (1) receiving a tissue sample of the abnormal tissue (e.g. tumor) from the patient; (2) dissociating the abnormal tissue (e.g. by mechanical and/or chemical process such as enzymatic disaggregation) to generate dissociated tissue; (3) separating the dissociated tissue into a plurality of samples; (4) culturing the plurality of samples to generate a plurality of mature cell clusters that replicate structures of the abnormal tissue from which they were sampled; (5) directing ultrasound signals with different ultrasound profiles to the plurality of mature cell clusters; (6) selecting the least-viable cell cluster from the plurality of mature cell clusters; (7) identifying the ultrasound profile corresponding to the least-viable cell cluster; (8) driving the identified ultrasound profile on ultrasonic emitters (e.g. emitters of transducers 120 and 130) to cause lysis of the abnormal tissue. In this way, the ultrasound profile that is most effective at achieving lysis for the actual target abnormal tissue may be identified and then used for therapy directed to the abnormal tissue. In some implementations, the mature cell clusters are brain cancer organoids.

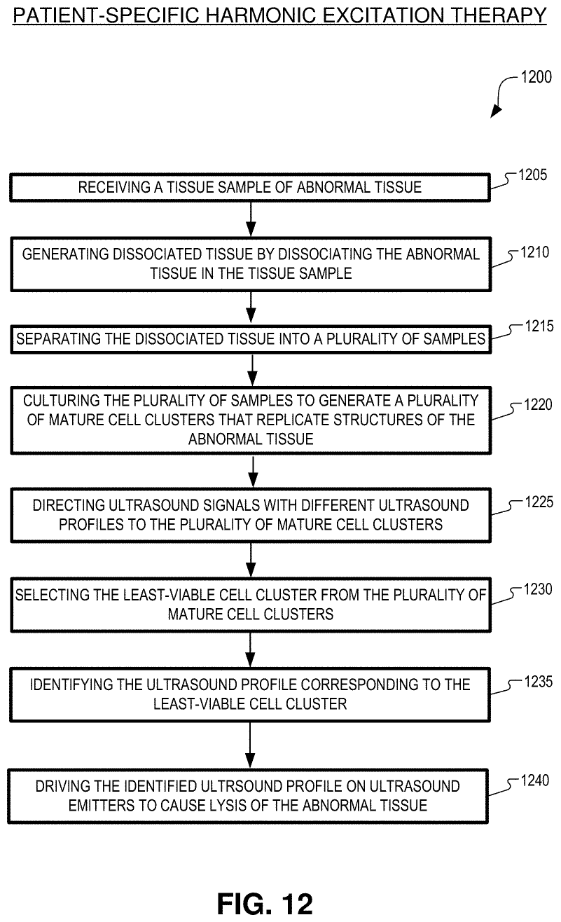

FIG. 12 illustrates an example flow chart illustrating an example process 1200 for patient-specific harmonic excitation therapy, in accordance with implementations of the disclosure. The order in which some or all of the process blocks appear in process 1200 should not be deemed limiting. Rather, one of ordinary skill in the art having the benefit of the present disclosure will understand that some of the process blocks may be executed in a variety of orders not illustrated, or even in parallel.

In process block 1205, a tissue sample of abnormal tissue is received.

In process block 1210, dissociated tissue is generated by dissociating the abnormal tissue in the tissue sample.

In process block 1215, the dissociated tissue is separated into a plurality of samples.

In process block 1220, the plurality of samples is cultured to generate a plurality of mature cell clusters that replicate structures of the abnormal tissue.

In process block 1225, ultrasound signals with different ultrasound profiles are directed to the plurality of mature cell clusters. The ultrasound profiles may vary with respect to frequency, pulse duration, and/or mechanical index.

In process block 1230, the least-viable cell cluster is selected from the plurality of mature cell clusters after the mature cell clusters receive their respective ultrasound signals.

In process block 1235, the ultrasound profile corresponding to the least-viable cell cluster is identified.

In process block 1240, the identified ultrasound profile (that caused the most cell death to the tumor sample) is driven onto ultrasound emitters to cause lysis of the abnormal tissue within the patient. In some contexts, a wearable device (e.g. device 150) that includes ultrasound emitters is programmed to direct their ultrasound signals having the identified ultrasound profile to the location of the tumor.

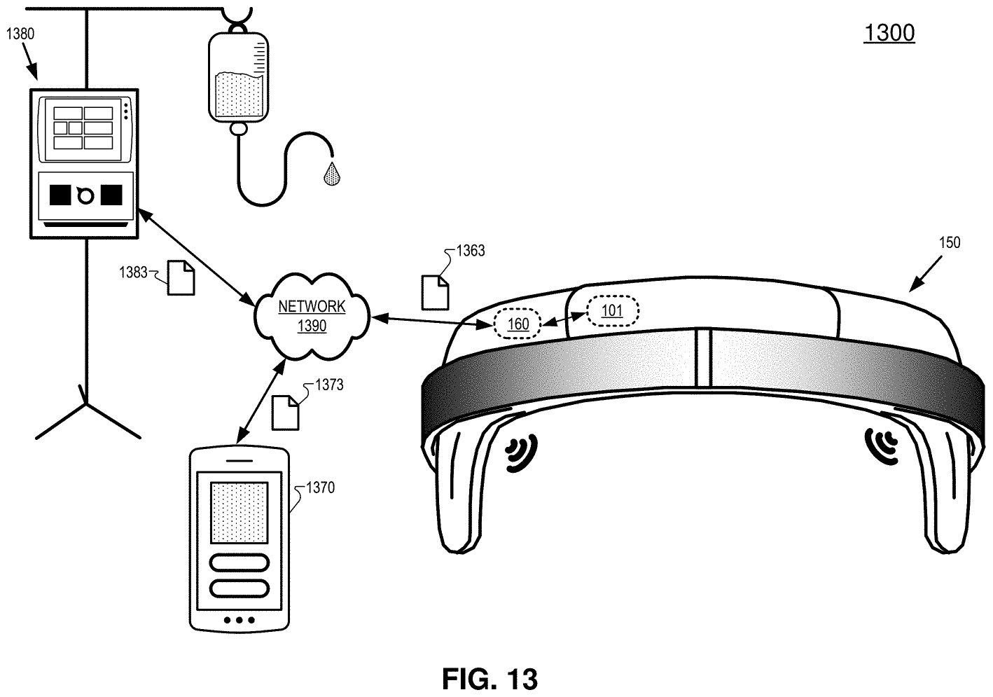

FIG. 13 illustrates an example therapy system 1300 that includes a wearable 150, a device 1370, and an example anti-tumor agent delivery device 1380, in accordance with implementations of the disclosure. Therapy system 1300 may be useful in pairing an anti-tumor agent with a specific ultrasound profile that causes lysis. Pairing the anti-tumor agent with the therapeutic ultrasound signal may increase neoantigen release from a tumor to prime an immune response from the patient.

Example anti-tumor agent delivery device 1380 is illustrated as including a controllable intravenous (IV) line that can control the rate of fluid of the IV line in response to a command 1383. In some implementations, an anti-tumor agent is delivered by way of the controllable IV line. In some implementations, the anti-tumor agent includes a small-molecule drug that can be dispensed by a device in response to command 1383. Examples of anti-tumor agent therapy may include (1) an antibody-based anti-tumor agent; (2) a monoclonal antibody; (3) bispecific antibody, (4) antibody-drug conjugate; (5) chimeric antigen receptor (CAR); (6) T cell therapy; (7) engineered T cell receptor (TCR) therapy; (8) adoptive T cell therapy; (9) natural killer (NK) cell therapy, or (10) dendritic cell therapy, for example. Systematic immunotherapies such as anti-PD-1, anti-PD-L1, anti-CTLA-4, anti-CD47, anti-sirp-alpha, and others may be included in an anti-tumor agent therapy.

Device 1370 may communicate with wearable 150 and anti-tumor agent delivery device 1380 via wired or wireless communications. Anti-tumor agent delivery device 1380 may communicate with wearable 150 and device 1370 via wired or wireless communications. Wearable 150 may communicate with device 1370 and anti-tumor agent delivery device 1380 via wired or wireless communications. In the illustrated implementation of FIG. 13, communications between wearable 150, anti-tumor agent delivery device 1380, and device 1370 are facilitated by network 1390. Network 1390 may be wired or wireless. Portions of network 1390 may be wired and portions of network 1390 may be wireless.

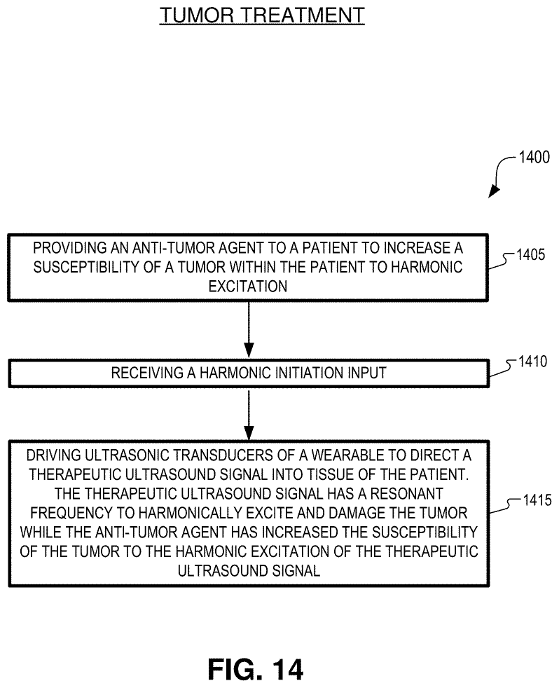

FIG. 14 illustrates an example flow chart illustrating an example tumor treatment process 1400, in accordance with implementations of the disclosure. The order in which some or all of the process blocks appear in process 1400 should not be deemed limiting. Rather, one of ordinary skill in the art having the benefit of the present disclosure will understand that some of the process blocks may be executed in a variety of orders not illustrated, or even in parallel. All or a portion of process 1400 may be executed within system 1300, for example.

In process block 1405, an anti-tumor agent is provided to a patient to increase a susceptibility of a tumor within the patient to harmonic excitation from ultrasound signals.

In process block 1410, a harmonic initiation input is received. The harmonic initiation input may be included in a message 1373 sent from device 1370. Device 1370 may be a computer or a mobile device (e.g. smartphone or tablet). A health care professional may interact with device 1370 in order to send the message 1373 that includes the harmonic initiation input. In some implementations, a patient interacts with device 1370 to send message 1373 that includes the harmonic initiation input. In some implementations, the harmonic initiation input is received by wearable 150 by a user interface (e.g. button or touchpad) included in wearable 150. The harmonic initiation input may also be included in message 1363 that is received by input interface 160. Network 1390 may generate message 1363 in response to receiving message 1373 from device 1370.

In process block 1415, ultrasound transducers (e.g. 120 and/or 130) are driven in response to receiving the harmonic initiation input. The ultrasound transducers direct a therapeutic ultrasound signal into tissue of the patient. The therapeutic ultrasound signal has a resonant ultrasound frequency to harmonically excite and damage the tumor while the anti-tumor agent has increased the susceptibility of the tumor to the harmonic excitation of the therapeutic ultrasound signal. Pairing the anti-tumor agent with the therapeutic ultrasound signal may increase neoantigen release from the tumor to prime an immune response from the patient.

Referring again to FIG. 13, wearable device 150 may receive an anti-tumor agent alert 1363 with input interface 160. As described previously, wearable 150 includes ultrasonic transducers (e.g. transducers 120 and 130 in FIG. 1C) configured to direct a therapeutic ultrasound signal into tissue (e.g. brain) of a patient. The anti-tumor agent alert 1363 is representative of an anti-tumor agent therapy being provided to the patient. The anti-tumor agent is an immunotherapy or a large molecule anti-tumor biologic agent. The anti-tumor agent therapy increases a susceptibility of a tumor within the patient to harmonic excitation caused by the therapeutic ultrasound signal(s).

In some implementations, wearable device 150 initiates the therapeutic ultrasound signal after a delay period so that the anti-tumor agent therapy has time to reach the tumor. In some implementations, the delay period is included in the anti-tumor agent alert 1363. In some implementations, a press of a software button on device 1370 (e.g. a mobile device) generates an anti-tumor agent therapy command 1383 for device 1380 to proceed with an anti-tumor agent therapy. Then, after some delay period, anti-tumor agent alert 1363 is transmitted by device 1370 to wearable 150 so that wearable 150 starts directing the therapeutic ultrasound signal(s) to the tumor. In some implementations, device 1380 is assisting in providing the anti-tumor agent to the patient and device 1380 transmits the anti-tumor agent alert 1363 to wearable 150 to notify wearable 150 that the anti-tumor agent therapy has been started. Hence, the pairing of the anti-tumor agent with the therapeutic ultrasound signal may be synchronized in various ways for the most beneficial therapy cadence.

In implementations of the disclosure, processing logic 101 is further configured to select the resonant ultrasound frequency of the therapeutic ultrasound signal in response to tumor-categorization data included in the anti-tumor agent alert 1363. By way of example, the tumor-categorization data may indicate the category of tumor that will be subject to the therapeutic ultrasound signal. Since the ideal therapeutic ultrasound signal to cause lysis of the tumor may be different for different categories of tumors, wearable 150 may adjust the resonant ultrasound frequency in response to the tumor-categorization data included in anti-tumor agent alert 1363. In an implementation, wearable 150 adjusts the resonant ultrasound frequency to 100 kHz in response to a first tumor-categorization in the tumor-categorization data and adjusts the resonant ultrasound frequency to 150 kHz in response to a second tumor-categorization in the tumor-categorization data.

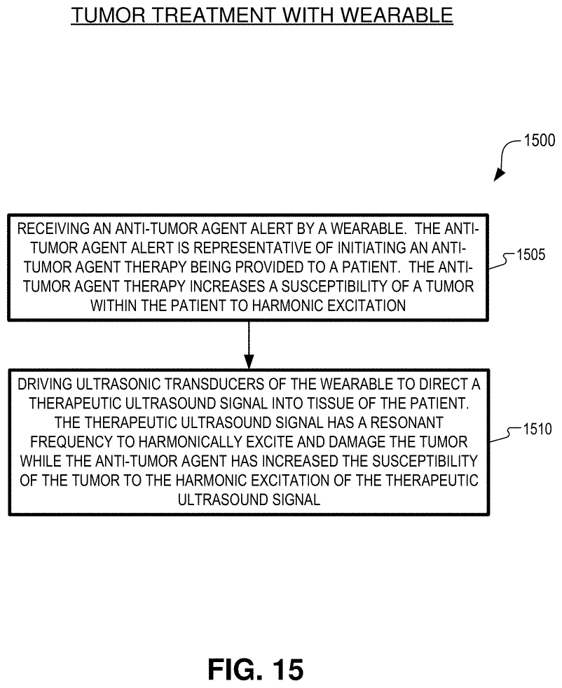

FIG. 15 illustrates an example flow chart illustrating an example tumor treatment process 1500, in accordance with implementations of the disclosure. The order in which some or all of the process blocks appear in process 1500 should not be deemed limiting. Rather, one of ordinary skill in the art having the benefit of the present disclosure will understand that some of the process blocks may be executed in a variety of orders not illustrated, or even in parallel. All or a portion of process 1500 may be executed by wearable 150, for example.

In process block 1505, an anti-tumor agent alert (e.g. alert 1363) is received by a wearable (e.g. wearable 150). The anti-tumor agent alert is representative of initiating an anti-tumor agent therapy being provided to a patient. The anti-tumor agent is provided to a patient to increase the susceptibility of a tumor within the patient to harmonic excitation from ultrasound signals.

In process block 1510, ultrasound transducers (e.g. 120 and/or 130) are driven in response to receiving the anti-tumor agent alert. The ultrasound transducers direct a therapeutic ultrasound signal into tissue of the patient. The therapeutic ultrasound signal has a resonant ultrasound frequency to harmonically excite and damage the tumor while the anti-tumor agent has increased the susceptibility of the tumor to the harmonic excitation of the therapeutic ultrasound signal. Pairing the anti-tumor agent with the therapeutic ultrasound signal may increase neoantigen release from the tumor to prime an immune response from the patient.

Some implementations of process 1500 further include selecting the resonant ultrasound frequency in response to tumor-categorization data included in the anti-tumor agent alert.

In some implementations, the resonant ultrasound frequency is delivered with a mechanical index of 95% or greater.

The term “processing logic” (e.g. 101) in this disclosure may include one or more processors, microprocessors, multi-core processors, Application-specific integrated circuits (ASIC), and/or Field Programmable Gate Arrays (FPGAs) to execute operations disclosed herein. In some embodiments, memories (not illustrated) are integrated into the processing logic to store instructions to execute operations and/or store data. Processing logic may also include analog or digital circuitry to perform the operations in accordance with embodiments of the disclosure.

A “memory” or “memories” described in this disclosure may include one or more volatile or non-volatile memory architectures. The “memory” or “memories” may be removable and non-removable media implemented in any method or technology for storage of information such as computer-readable instructions, data structures, program modules, or other data. Example memory technologies may include RAM, ROM, EEPROM, flash memory, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device.

Networks may include any network or network system such as, but not limited to, the following: a peer-to-peer network; a Local Area Network (LAN); a Wide Area Network (WAN); a public network, such as the Internet; a private network; a cellular network; a wireless network; a wired network; a wireless and wired combination network; and a satellite network.

Communication channels (e.g. X1 and X2) may include or be routed through one or more wired or wireless communication utilizing IEEE 802.11 protocols, short-range wireless, SPI (Serial Peripheral Interface), I2C (Inter-Integrated Circuit), USB (Universal Serial Port), CAN (Controller Area Network), cellular data protocols (e.g. 3G, 4G, LTE, 5G), optical communication networks, Internet Service Providers (ISPs), a peer-to-peer network, a Local Area Network (LAN), a Wide Area Network (WAN), a public network (e.g. “the Internet”), a private network, a satellite network, or otherwise.

A computing device may include a desktop computer, a laptop computer, a tablet, a phablet, a smartphone, a feature phone, a server computer, or otherwise. A server computer may be located remotely in a data center or be stored locally.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible non-transitory machine-readable storage medium includes any mechanism that provides (i.e., stores) information in a form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A therapy system comprising:
a wearable article including one or more ultrasonic transducers; and
processing logic configured to drive the one or more ultrasonic transducers to direct a therapeutic ultrasound signal into tissue brought into contact with the ultrasonic transducers, wherein the therapeutic ultrasound signal has a resonant ultrasound frequency to harmonically excite and damage abnormal tissue but not harmonically excite healthy tissue, wherein the one or more ultrasonic transducers are configured to direct the therapeutic ultrasound signal to propagate through the abnormal tissue and the healthy tissue.

2. The therapy system of claim 1, wherein the processing logic is configured to drive the ultrasonic transducers to scan the therapeutic ultrasound signal through a brain to provide the therapeutic ultrasound signal to substantially all of the tissue in the brain, wherein driving the ultrasonic transducers includes driving the ultrasonic transducers with beam forming pattern signals to achieve the scan.

3. The therapy system of claim 1, wherein the resonant ultrasound frequency is between 50 kHz and 1 MHz.

4. The therapy system of claim 1, wherein the resonant ultrasound frequency is between 75 kHz and 85 kHz.

5. The therapy system of claim 1, wherein the resonant ultrasound frequency is between 95 kHz and 105 kHz.

6. The therapy system of claim 1, wherein the resonant ultrasound frequency is between 145 kHz and 155 kHz.

7. The therapy system of claim 1, wherein the therapeutic ultrasound signal is directed into the tissue in bursts that are 30 ms or longer.

8. The therapy system of claim 1, wherein the processing logic is further configured to:
receive a medical image;
identify the abnormal tissue from the healthy tissue included in the medical image; and
determine the resonant ultrasound frequency of the abnormal tissue from the medical image.

9. The therapy system of claim 1, wherein the therapeutic ultrasound signal is directed into the tissue in bursts of approximately 40 ms.

10. A computer-implemented therapy method comprising:
receiving a medical image;
identifying abnormal tissue from healthy tissue included in the medical image;
determining a resonant ultrasound frequency of the abnormal tissue; and
directing an ultrasound transducer to emit a therapeutic ultrasound signal to the abnormal tissue at the resonant ultrasound frequency to harmonically excite the abnormal tissue but not harmonically excite the healthy tissue.

11. The therapy method of claim 10, wherein the therapeutic ultrasound signal propagates through the healthy tissue and the abnormal tissue.

12. The therapy method of claim 10, wherein determining the resonant ultrasound frequency of the abnormal tissue includes identifying the abnormal tissue as a particular tumor type from a group of tumor types and assigning a particular ultrasound frequency as the resonant ultrasound frequency, wherein the particular ultrasound frequency is associated with harmonic excitation of the particular tumor type.

13. The therapy method of claim 10, wherein the medical image is one of an MRI or a CT scan.

14. The therapy method of claim 10, wherein the resonant ultrasound frequency is approximately 80 kHz.

15. The therapy method of claim 10, wherein the resonant ultrasound frequency is approximately 100 kHz.

16. The therapy method of claim 10, wherein the resonant ultrasound frequency is approximately 150 kHz.

17. The therapy method of claim 10, wherein the therapeutic ultrasound signal is directed in bursts that are 30 ms or longer.

18. The therapy method of claim 10, wherein directing the ultrasound transducer to emit the therapeutic ultrasound signal includes driving a plurality of the ultrasonic transducers with beam forming pattern signals to direct the therapeutic ultrasound signal to the abnormal tissue identified from the medical image.

19. The therapy method of claim 10, wherein the therapeutic ultrasound signal is directed in bursts of approximately 40 ms.

20. A computer-implemented method comprising:
directing ultrasound signals with different ultrasound profiles to a plurality of mature cell clusters, wherein the mature cell clusters are cultured from abnormal tissue;
selecting a least-viable cell cluster from the plurality of mature cell clusters, wherein the selecting is subsequent to directing the ultrasound signals with the different ultrasound profiles to the plurality of mature cell clusters;
identifying the ultrasound profile corresponding to the least-viable cell cluster; and
driving the identified ultrasound profile on ultrasound emitters of a wearable device to cause lysis of the abnormal tissue.
